(19) United States
(12) Patent Application Publication
(10) Pub. No.: US 2012/0330154 A1
(43) Pub. Date: Dec. 27, 2012

(54) MEDICAL EXAMINATION AND/OR TREATMENT DEVICE

(76) Inventors: Paul Beasley, Abingdon (GB); Oliver Heid, Erlangen (DE); Timothy Hughes, Erlangen (DE)

(21) Appl. No.: 13/582,740

(22) PCT Filed: Mar. 4, 2011
(86) PCT No.: PCT/EP11/53256

§ 371 Date: Sep. 4, 2012

(30) Foreign Application Priority Data
Mar. 4, 2010 (DE) 10 2010 010 192.3

(51) Int. Cl. A61B 6/00; A61N 5/10

(52) U.S. Cl. 600/436; 600/1

(57) ABSTRACT

A medical examination and/or treatment device for performing image acquisitions and/or radiation- or instrument-based treatments in an examination or treatment region is provided, The device may include an image acquisition means and/or a treatment means and a patient table, wherein a detection means for determining the position of a patient located on the patient table is provided, the detection means including a radiation emitter which irradiates the patient in at least one region and emits terahertz radiation, at least one radiation receiver detecting reflected terahertz radiation, and a processing means processing the receiver signals supplied by the radiation receiver, wherein the processing means can generate an image showing the surface of the irradiated patient region on the basis of the receiver signals and the position of the patient region relative to the examination or treatment region can be determined on the basis of said image.

### CROSS-REFERENCE TO RELATED APPLICATIONS

This application is a U.S. National Stage Application of International Application No. PCT/EP2011/053256 filed Mar. 4, 2011, which designates the United States of America, and claims priority to DE Patent Application No. 10 2010 010 192.3 filed Mar. 4, 2010. The contents of which are hereby incorporated by reference in their entirety.

### TECHNICAL FIELD

This disclosure relates to a medical examination and/or treatment device for carrying out imaging and/or radiation- or instrument-based treatments in an examination or treatment region, comprising an imaging means and/or a treatment means as well as a patient table.

### BACKGROUND

In the scope of carrying out medically indicated examinations or treatments, imaging is often carried out with a wide variety of modalities such as X-ray devices, ultrasound devices, PET devices and the like, or interventional treatments, for example with robot control. To this end, it is necessary to ensure that the examination or treatment, i.e. for example the imaging or the interventional operation, is actually carried out in the correct body region, that is to say the patient is positioned correctly with respect to the imaging or treatment means. To this end, above all when prolonged examinations or treatments are involved, the patient is often fixed, although inter alia this is uncomfortable for the patient.

### SUMMARY

In one embodiment, a medical examination and/or treatment device for carrying out imaging and/or radiation- or instrument-based treatments in an examination or treatment region comprises: an imaging means and/or a treatment means as well as a patient table, wherein a detection means is provided for determining the position of a patient lying on the patient table, comprising a radiation emitter which emits terahertz radiation and irradiates the patient in at least one region, at least one radiation receiver which detects reflected terahertz radiation, and a processing means which processes the receiver signals delivered by the radiation receiver, a representation showing the surface of the irradiated patient region being generatable by the processing means with the aid of the receiver signals and the position of the patient region relative to the examination or treatment region being determinable therefrom.

In a further embodiment, two or more radiation receivers arranged at different positions are provided, a 3D representation of the patient region being generatable by the processing means with the aid of the different receiver signals and the position of the patient region relative to the examination or treatment region being determinable from the 3D representation. In a further embodiment, the radiation

emitter and radiation receiver or receivers are arranged on the device itself or next to it. In a further embodiment, at least one marking which at least partially absorbs or reflects the terahertz radiation during irradiation by the radiation emitter is provided in or on the patient table, the position of the marking shown in the generated representation in the coordinate system of the device, and the position of the patient region relative to the marking, being determinable by the processing means and the position of the patient region relative to the examination or treatment region being determinable from the two position information items. In a further embodiment, a plurality of markings are arranged distributed in or on the patient table. In a further embodiment, a marking has a defined 3D geometry.

In another embodiment, a method for determining the position of a patient relative to an examination and/or treatment region of an imaging means and/or a treatment means of a medical examination and/or treatment device is provided, wherein a region of the patient is irradiated by a radiation emitter which emits terahertz radiation and reflected terahertz radiation is detected by means of at least one radiation receiver, and in that a representation showing the surface of the irradiated patient region is generated by means of a processing means with the aid of the receiver signals delivered by the radiation receiver and the position of the patient region relative to the examination and/or treatment region is determined therefrom.

In a further embodiment, separate receiver signals from two or more radiation receivers arranged at different positions are delivered to the processing means, a 3D representation of the patient region being generated by the processing means with the aid of the different receiver signals and the position of the patient region relative to the examination and/or treatment region being determined from the 3D representation. In a further embodiment, at least a part of a patient table carrying the patient, in or on which at least one marking which at least partially absorbs or reflects the terahertz radiation during irradiation by the radiation emitter is provided, is also irradiated by the radiation emitter, the position of the marking shown in the generated representation in the coordinate system of the device, and the position of the patient region relative to the marking, being determined by the processing means and the position of the patient region relative to the examination and/or treatment region being determined from the two position information items.

### BRIEF DESCRIPTION OF THE DRAWINGS

Example embodiments will be explained in more detail below with reference to figures, in which:

FIG. 1 shows a schematic representation of a medical examination and/or treatment device according to a first embodiment, and

FIG. 2 shows a medical examination and/or treatment device according to a second embodiment.

### DETAILED DESCRIPTION

Some embodiments provide a medical examination and/or treatment device which allows position monitoring of the patient in an improved way.

For example, in some embodiments, a medical examination and/or treatment device may include a detection means for determining the position of a patient lying on the patient table, comprising a radiation emitter which emits terahertz radiation and irradiates the patient in at least one region, at least one radiation receiver which detects reflected terahertz radiation, and a processing means which processes the receiver signals delivered by the radiation receiver, a representation showing the surface of the irradiated patient region being generatable by the processing means with the aid of the receiver signals and the position of the patient region relative to the examination or treatment region being determinable therefrom.

In some embodiments, the examination and/or treatment device may use a position detection means which operates on an emitter-receiver basis. The emitter emits terahertz radiation, which is reflected by the patient, the receiver detects the reflected terahertz radiation and delivers corresponding receiver signals which are processed further in a processing means. The terahertz radiation, which covers a frequency range of about 150 GHz-10 THz, particularly advantageously does not penetrate into the patient, or penetrates only insubstantially, and is thus reflected from the surface or from near the surface. The reflected signal, the frequency or amplitude of which is changed due to the reflection, consequently images the irradiated surface of the patient. From the receiver signals, the processing means can consequently generate terahertz images with the aid of which the processing means optionally furthermore carries out image processing steps such as one or more segmentations (using suitable algorithms such as edge- or region-based algorithms), pattern recognition methods and the like, in order to automatically determine the exact patient region geometry from the surface image. From this, information can consequently be acquired about the location, or position, of the patient with respect to the examination or treatment region, the position of which is known. With the aid of the surface representation, the processing means determines what the patient region looks like, or what it is, and checks whether it is the region defined by the user, which is intended to be examined or treated. Knowing what the region is, it is consequently possible to ascertain whether or not the position of the patient is correct. The surface of the patient region is highly suitable for the position determination, since it delivers a very accurate image of the patient. In particular, using the terahertz radiation does not entail any radiation doses, such as occur for example if, when carrying out X-ray recordings, an X-ray image is previously taken for the position determination. Another advantage is that the terahertz images can of course be recorded continuously, so that continuous position monitoring is possible. It is furthermore advantageous that the clothing worn by the patient is transparent for the terahertz radiation, and therefore does not cause disturbance, so that the surface images which are expedient according to embodiments disclosed herein can be recorded in each case.

Furthermore, any movements or position changes of the patient can be detected by simple comparison of the continuously recorded surface images, and reacted to if necessary.

The patient position which is determined (if it is determined directly in the processing means) or the surface image which is generated, or any information extracted therefrom regarding the patient geometry, is in each case delivered to the control unit of the device, which controls the operation of the imaging means and/or the treatment means. If a position comparison reveals that the patient is positioned correctly relative to the examination and/or treatment region, the examination or treatment may begin, or in the case of continuous monitoring it may be continued. If incorrect positioning is revealed, then, if the treatment has not yet begun, corresponding measures may be carried out to correct the position or, if the treatment is already ongoing, measures for repositioning or if need be even temporary interruption of the examination and/or treatment may be carried out.

According to one embodiment, two or more radiation receivers arranged at different positions are used, a 3D representation of the patient region being generatable by the processing means with the aid of the different receiver signals and the position of the patient region relative to the examination or treatment region being determinable from the 3D representation. While two 3D surface images can be generated when using one radiation emitter and one radiation receiver, the use of one radiation emitter and at least two radiation receivers arranged at different positions makes it possible to generate, or reconstruct, 3D surface representations of the patient region. A stereographic arrangement of the radiation receivers is thus proposed, in order to generate a 3D surface representation by using stereographic image processing techniques. A three-dimensional representation permits even better geometry determination and position acquisition.

The radiation emitter and radiation receiver or receivers may in this case be arranged on the device itself, although it is also conceivable to position the emitter and the receivers next to the device. It is of course also conceivable, for example, to position the radiation emitter on the device and the receivers externally to the device, or vice versa. Any arrangement is possible so long as good region irradiation and good reception of the reflected terahertz radiation are possible.

In order to determine the absolute patient table position, it is conventional to determine the table movement for example by means of mechanical counters, which record the displacement length of the table, or electrical position measurement means. In order then to be able to determine the patient position straightforwardly in the form of absolute coordinates in the coordinate system of the examination and/or treatment device as well, according to one embodiment at least one marking which at least partially absorbs or reflects the terahertz radiation during irradiation by the radiation emitter is provided in or on the patient table, the position of the marking shown in the generated representation in the coordinate system of the device, and the position of the patient region relative to the marking, being determinable by the processing means and the position of the patient region relative to the examination or treatment region being determinable from the two position information items. In some embodiments, at least one marking, which has a determined geometrical structure and absorbs or reflects terahertz radiation, is embedded in the patient table. The image recorded by the terahertz radiation receiver shows the patient region and the patient table, at least locally, with the absorbent or reflective marking. The marking is imaged very accurately and with defined contrast in the image, and can be determined by suitable image processing means (segmentation, pattern recognition, etc.). By means of suitable further image processing possibilities, the relative position of the patient region with respect to the marking can then furthermore be determined. Since the position of the patient table in the coordinate system of the examination and/or treatment device is known, it is then possible to calculate the position of the patient in the coordinate system of the examination and/or treatment device from the additional knowledge of the relative patient region position with respect to the marking, which thus constitutes a reference point, and from the position of the marking in space per se. Since the position of the marking does not change during the examination or treatment, as the table is conventionally not moved, the marking therefore constitutes a reference point in the coordinate system of the examination and/or treatment device, which does not change during the process and with respect to which the patient position determination can be carried out.

It is expedient for a plurality of markings to be distributed in or on the patient table, these preferably being provided on the edge, so that they can also be irradiated and consequently imaged in the image. A marking preferably has a defined 3D geometry, so that any table adjustments, i.e. tilting or rotation, can also be detected with the aid of the marking geometry shown in the image.

Any desired receiver which delivers a signal when recording terahertz radiation may be used as the radiation receiver, but also suitable cameras which are sensitive in this frequency range.

Besides the medical examination and/or treatment device itself, some embodiments relate to a method for determining the position of a patient relative to an examination and/or treatment region of an imaging means and/or a treatment means of a medical examination and/or treatment device. The method is characterized in that a region of the patient is irradiated by a radiation emitter which emits terahertz radiation and reflected terahertz radiation is detected by means of a radiation receiver, and in that a representation showing the surface of the irradiated patient region is generated by means of a processing means with the aid of the receiver signals delivered by the receiver and the position of the patient region relative to the examination and/or treatment region is determined therefrom.

If two or more radiation receivers, which are arranged at different positions and deliver separate receiver signals, are used in the scope of the method disclosed herein, then with this stereoscopic receiver arrangement, owing to the position-related different surface representations of the respective receivers, a 3D representation of the patient region, with the aid of which the position determination of the patient region is carried out, can be generated by suitable stereoscopic processing methods.

If at least a part of the patient table, in or on which at least one marking which at least partially absorbs or reflects the terahertz radiation during irradiation by the radiation emitter is provided, is also irradiated by the radiation emitter, then by the processing means it is possible to determine the position of the marking shown in the generated representation in the coordinate system of the device, and the position of the patient region relative to the marking, and to determine the position of the patient region relative to the examination and/or treatment region from the two position information items. This is possible both when using only one radiation receiver and when using a plurality of radiation receivers positioned in a distributed way in a stereoscopic arrangement.

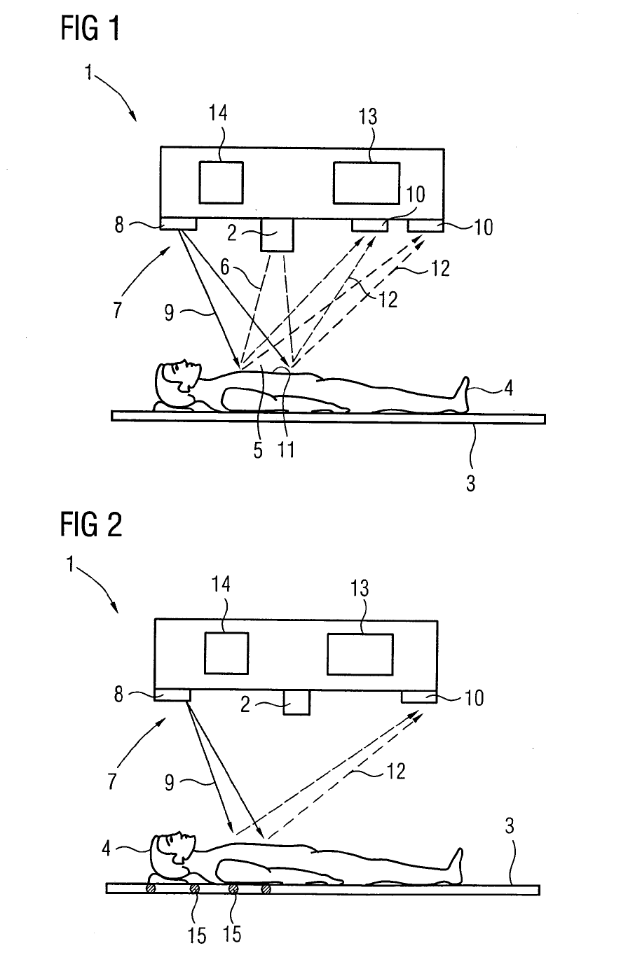

FIG. 1 shows a medical examination and/or treatment device 1 according to one embodiment in the form of a purely schematic representation. The examination and/or treatment device 1 in the example shown has, by way of example, an X-ray imaging means 2 comprising for example the X-ray source shown here and an X-ray detector, not shown in any more detail here. By means of this X-ray imaging device, it is possible to record X-ray images of the patient 4 lying on a patient table 3.

In order now to determine the spatial position of the patient and therefore his or her location relative to the examination or treatment region 5, which is defined here for example by the beam cone 6 of the X-ray imaging device 2, a detection means 7 is provided which allows exact position determination by recording and evaluating surface images of the patient 4. In the example shown, the detection means comprises a radiation emitter 8 which emits terahertz radiation 9, represented by the beam fan, onto the patient 4. Two radiation receivers 10 are furthermore provided in the example shown, and are arranged offset with respect to one another. The terahertz radiation applied by the radiation emitter 8 is reflected by the surface 11 of the patient 4, that is to say depending on the wavelength it does not penetrate or penetrates only insubstantially into the patient. The reflected terahertz radiation 12 is recorded by the respective radiation receiver 10. Each radiation receiver 10 consequently delivers corresponding receiver signals to a processing means 13, which is part of the detection means 7, processes the receiver signals and determines therefrom surface images which highly accurately image the surface 11 of the patient which has been exposed to the emitted tera radiation 9. Since two radiation receivers 10 arranged mutually offset are provided, i.e. the arrangement is a stereoscopic arrangement, different images are readily detected, or generated, by the two radiation receivers 10. With the aid of the minor image differences of two simultaneously recorded images, a three-dimensional representation of the irradiated surface can then be generated, i.e. reconstructed.

By suitable image processing algorithms such as segmentation algorithms, pattern recognition algorithms etc., the processing means 13 then determines the exact geometry of the surface. From this geometrical information relating to this recorded patient table, the control unit 14 which controls the operation of the examination and/or treatment device, i.e. here for example the operation of the X-ray imaging device 2, can then in turn determine the position of the patient relative to the examination and/or treatment region 5, and can thus identify from the surface information, or geometrical information, whether the previously defined desired region which is to be examined or treated actually lies in the examination and/or treatment region 5, or whether it is still another region which has a different geometry, which does not correspond to the region geometry belonging to the region to be treated. If the evaluation of the geometrical information from the surface representation reveals that the correct patient region is in the examination region 5, the position of which is known, then the examination or treatment may be started. If it reveals that the positioning is still not correct, the patient table 3 may be correspondingly moved in order to position the patient 4 correctly.

Here, it should be pointed out that the position determination may be carried out continuously, that is to say position monitoring may also continue to be carried out continuously during the examination or treatment. As soon as a patient movement causes displacement of the patient region actually to be examined or treated relative to the examination region 5, the control unit 14 can immediately take action and, for example, carry out correction of the table or intervene in the examination or treatment process.

FIG. 2 shows a second embodiment of an examination and/or treatment device 1 according to one embodiment, the same references being used for components which are the same. A patient table 3 is again provided, on which a patient 4 lies, as well as a detection means 7 comprising a radiation emitter 8 which emits terahertz radiation, and, in the example shown, only one radiation receiver 10 which receives the reflected terahertz radiation 12. The processing means 13 in turn generates a surface image which is evaluated with respect to the geometry of the irradiated patient region, and the corresponding geometrical information is delivered to the control unit 14 which determines the position therefrom. In this example, only one radiation receiver 10 is provided, for which reason the geometry determination is carried out with the aid of a 2D image. It is of course also conceivable to provide two stereoscopically arranged radiation receivers 10 in this case, and likewise of course it is possible to provide only one radiation receiver 10 in the exemplary embodiment according to FIG. 1.

In this configuration, a plurality of markings 15 are provided on or in the patient table 3, which either absorb or reflect the terahertz radiation 9 which strikes them when there is a corresponding configuration of the beam cone. The reflected terahertz radiation 12, which is recorded by the radiation receiver 10, consequently also images the region of the one or more irradiated markings 15, that is to say the surface image representation generated in the processing means 13 also shows the markings. Since they have either an absorbing or reflecting effect, these can be detected very accurately and with sharp contrast.

Since the table 3 is motor-driven, it is readily possible to determine the absolute position of the markings 15 in the coordinate system of the examination and/or treatment device 1. That is to say, its absolute position coordinates are known by the processing means 13. The processing means 13 is then capable, by suitable image processing algorithms, on the one hand to detect the markings exactly in the image, for example by suitable edge detection algorithms or region-based algorithms or the like. Furthermore, the geometry of the patient is determined, as well as the relative position of the patient region, that is to say the geometry with respect to the marking or markings 15 shown in the surface representation. All the information is delivered to the control unit 14.

If the position of the patient 4 on the table subsequently changes in the course of the examination or treatment, even though this table remains fixed in position, then there will necessarily be a change in the relative position of the patient region, i.e. the recorded geometry, with respect to the marking or markings 15 likewise shown in the image. By means of this, the relative position of the geometry with respect to the positionally fixed markings can thus be determined continuously, and any position changes can immediately be detected and processed by the control unit 14, and corrective actions may optionally be implemented.

The known position coordinates of the markings 15 in the coordinate system of the examination and/or treatment device 1 now also make it possible to determine accurate position coordinates of the determined geometry of the patient region, i.e. in the end of the patient himself or herself, in the coordinate system of the examination and/or treatment device. This means that the exact spatial position of the patient, or of the scanned patient region, is also known in absolute coordinates of the coordinate system of the examination and/or treatment device. If a movement subsequently takes place, then since it is also immediately detectable from the image representations in its extent, it can be converted exactly into corresponding coordinate changes, from which for example corresponding control signals may be sent by the control unit 14 to the patient table, which is then moved accordingly in order to reposition the patient region to be treated, etc.

### LIST OF REFERENCES

1 medical examination and/or treatment device
2 X-ray imaging means
3 patient table
4 patient
5 examination or treatment region
6 beam cone
7 detection means
8 radiation emitter
9 terahertz radiation
10 radiation receiver
11 surface
12 terahertz radiation
13 processing means
14 control unit
15 markings

What is claimed is:

1. A medical examination and/or treatment device for carrying out imaging and/or radiation- or instrument-based treatments in an examination or treatment region, the device comprising:
an imaging means and/or a treatment means,
a detection system configured to determine the position of a patient lying on a patient table, comprising:
a radiation emitter configured to emit terahertz radiation and irradiate the patient in at least one region,
at least one radiation receiver configured to detect reflected terahertz radiation, and
a processing device configured to:
process the receiver signals delivered by the radiation receiver,
generate a representation showing the surface of the irradiated patient region based on the receiver signals, and
automatically determine a position of the patient region relative to the examination or treatment region based at least on the generated representation.

2. The device of claim 1, comprising two or more radiation receivers arranged at different positions,
wherein the processing device is configured to:
generate a 3D representation of the patient region based on the different receiver signals, and
determine the position of the patient region relative to the examination or treatment region based on the 3D representation.

3. The device of claim 1, wherein the radiation emitter and the at least one radiation receiver are arranged on or adjacent to the device.

4. The device of claim 1, comprising at least one marking that at least partially absorbs or reflects the terahertz radiation during irradiation by the radiation emitter,
wherein the position of the marking is indicated in the generated representation, and
wherein the processing means is configured to:
determine a position of the patient region relative to the marking, and
determine the position of the patient region relative to the examination or treatment region based on (a) the position of the marking indicated in the generated representation and (b) the determined position of the patient region relative to the marking.

5. The device of claim 4, wherein a plurality of markings are arranged distributed in or on the patient table.

6. The device of claim 4, wherein a marking has a defined 3D geometry.

7. A method for determining the position of a patient relative to an examination and/or treatment region of an imaging device and/or a treatment device of a medical examination and/or treatment device, the method comprising:
irradiating a region of the patient using a radiation emitter that emits terahertz radiation,
detecting reflected terahertz radiation using at least one radiation receiver,
using a processing device, generating a representation showing the surface of the irradiated patient based on the receiver signals delivered by the radiation receiver, and
using the processing device, determining a position of the patient region relative to the examination and/or treatment region based on the generated representation.

8. The method of claim 7, comprising:
delivering to the processing device separate receiver signals from two or more radiation receivers arranged at different positions,
using the processing device, generating a 3D representation of the patient region based on the different receiver signals, and
using the processing device, determining the position of the patient region relative to the examination and/or treatment region based on the 3D representation.

9. The method of claim 7, comprising:
irradiating at least one marking in or on a patient table, the at least one marking least partially absorbing or reflecting the terahertz radiation during irradiation by the radiation emitter, such that the position of the marking is indicated in the generated representation
using the processing means, determining a position of the patient region relative to the marking, and
using the processing means, determining the position of the patient region relative to the examination or treatment region based on (a) the position of the marking indicated in the generated, representation and (b) the determined position of the patient region relative to the marking.

10. The method of claim 9, wherein a plurality of markings are arranged distributed in or on the patient table.

11. The method of claim 9, wherein a marking has a defined 3D geometry.
